(19) United States
(12) Patent Application Publication
(10) Pub. No.: US 2026/0260156 A1
(43) Pub. Date: Sep. 3, 2026

(54) METHODS, SYSTEMS, AND COMPUTER READABLE MEDIA FOR STORING AND PROCESSING ULTRASOUND AUDIO DATA

(71) Applicant: THE JOHNS HOPKINS UNIVERSITY, Baltimore, MD (US)

(72) Inventors: Max KERENSKY, Baltimore, MD (US); Amir MANBACHI, Baltimore, MD (US); Kelley KEMPSKI LEADINGHAM, Baltimore, MD (US); Nitish THAKOR, Laurel, MD (US); Nicholas THEODORE, Baltimore, MD (US)

(21) Appl. No.: 18/881,936

(22) PCT Filed: Jul. 19, 2023
(86) PCT No.: PCT/US2023/028138

§ 371 Date: Jan. 7, 2025

Related U.S. Application Data
(60) Provisional application No. 63/390,713, filed on Jul. 20, 2022.

(51) Int. Cl. G06N 20/00

(52) U.S. Cl. CPC G06N 20/00; A61B 8/00; A61B 5/72; A61B 8/06

(57) ABSTRACT

A method for diagnosing a physiological state of a patient includes capturing ultrasound audio data in the patient using a sensor. The method also includes processing the ultrasound audio data to produce processed ultrasound audio data. The method also includes generating an output based at least partially upon the processed ultrasound audio data. The output provides the physiological state of the patient. The physiological state of the patient includes a certainty of an existence of a non-healthy region in the patient at a plurality of different times and a location of the non-healthy region in the patient at the different times.

### CROSS-REFERENCE TO RELATED APPLICATIONS

This application is the national stage entry of International Patent Application No. PCT/US2023/028138, filed on Jul. 19, 2023, and published as WO 2024/020093 A2 on Jan. 25, 2024, which claims the benefit of U.S. Provisional Patent Application No. 63/390,713, filed on Jul. 20, 2022, which are hereby incorporated by reference in their entireties.

### GOVERNMENT SUPPORT

This invention was made with Government support under Grant No. N66001-20-2-4075, awarded by the Department of the Navy and funded by the Defense Advanced Research Projects Agency (DARPA). The Government has certain rights in the invention.

### FIELD OF THE DISCLOSURE

The present disclosure relates generally to systems and methods for processing ultrasound audio data. More particularly, the present disclosure relates to systems and methods for processing ultrasound audio data to diagnose (e.g., evaluate, predict, inform, track, etc.) a physiological state of a patient.

### BACKGROUND OF THE DISCLOSURE

With the ability to quantify blood flow, spectral Doppler ultrasound has become an integral component of diagnostic capabilities. Conventional ultrasound systems can plot the blood velocity within a vessel over time, yielding insights into potential underlying conditions or physiological states. Though the fundamental basis of Doppler technology is to detect motion-induced changes to the propagation of sound, conventional ultrasound scanners do not store the real-time audio output associated with the Doppler shift. Instead, these scanners process and lose data via production of visual displays including charts. This is unfortunate for two reasons. First, sonographers are trained to use this audio during evaluations for feedback but are unable to replay it after the scan. Second, sound-based method of blood flow diagnostics is prevented along with the potential for data loss.

### SUMMARY

A method for diagnosing a physiological state of a patient is disclosed. The method includes capturing ultrasound audio data in the patient using a sensor. The method also includes processing the ultrasound audio data to produce processed ultrasound audio data. The method also includes generating an output based at least partially upon the processed ultrasound audio data. The output provides the physiological state of the patient. The physiological state of the patient includes a certainty of an existence of a non-healthy region in the patient at a plurality of different times and a location of the non-healthy region in the patient at the different times.

In another embodiment, the method includes capturing ultrasound audio data in the patient using a sensor. The ultrasound audio data includes spectral Doppler audio data of an organ in the patient. The method also includes processing the ultrasound audio data to produce processed ultrasound audio data. Processing the ultrasound audio data includes performing a spatial transformation and/or a temporal transformation on the ultrasound audio data to produce a graph. The method also includes analyzing acoustic biomarkers in the processed ultrasound audio data to produce analyzed ultrasound audio data. The acoustic biomarkers are analyzed by combining the processed ultrasound audio data from different times. The method also includes generating images of the organ the different times. The method also includes performing machine learning (ML) on the analyzed ultrasound audio data to produce ML ultrasound audio data. The ML includes classification, grading, ranking, uniform manifold approximation and projection (UMAP), t-distributed stochastic neighbor embedding (t-SNE), or a combination thereof. The method also includes generating an output based at least partially upon the ML ultrasound audio data. The output provides the physiological state of the patient. The physiological state of the patient includes a certainty of an existence of a non-healthy region at the different times and a location of the non-healthy region at the different times.

In yet another embodiment, the method includes capturing ultrasound audio data from the patient using a sensor. The ultrasound audio data includes spectral Doppler audio data. The method also includes processing the ultrasound audio data to produce processed ultrasound audio data. Processing the ultrasound audio data includes performing a spatial transformation and a temporal transformation on the ultrasound audio data to produce a graph in a time-frequency domain. The graph includes an envelope of the ultrasound audio data and speckles below the envelope. The speckles represent regions of high-density, signal origin isolation in the ultrasound audio data. The method also includes analyzing acoustic biomarkers in the graph to produce analyzed ultrasound audio data. The acoustic biomarkers are analyzed in the envelope and in the speckles below the envelope. The acoustic biomarkers are analyzed by combining the processed ultrasound audio data from different times, different locations in a same organ, different organs, or a combination thereof. The method also includes generating images of the same organ and/or the different organs at the different times to show a disruption. The method also includes performing machine learning (ML) on the images to produce ML ultrasound audio data. The ML includes classification, grading, ranking, uniform manifold approximation and projection (UMAP), t-distributed stochastic neighbor embedding (t-SNE), or a combination thereof. The method also includes generating an output based at least partially upon the ML ultrasound audio data. The output provides the physiological state of the patient. The the physiological state of the patient includes a certainty of an existence of the disruption at the different times, a size of the disruption at the different times, the location of the disruption at the different times, and a trend or a progression of the disruption at the different times and at future times.

### BRIEF DESCRIPTION OF THE FIGURES

FIG. 1 illustrates a schematic view of a system for capturing and processing ultrasound audio data, according to an embodiment.

FIG. 2 illustrates a flowchart of a method for diagnosing a physiological state of a patient, according to an embodiment.

FIG. 3 illustrates a flowchart of another method for diagnosing the physiological state of the patient, according to an embodiment.

FIG. 4 illustrates a graph showing spectral Doppler audio data, according to an embodiment.

FIG. 5 illustrates a graph (e.g., periodogram) showing the spectral Doppler audio data after a spatial and/or temporal frequency transformation, according to an embodiment.

FIGS. 6A and 6B illustrate a plurality of graphs (e.g., periodograms) corresponding to different times, different organs (e.g., vessels), and/or different locations (e.g., in the same organ), according to an embodiment.

FIG. 7A illustrates an output (e.g., audio-frequency graph) for a healthy patient, and FIG. 7B illustrates an output (e.g., audio-frequency graph) for a non-healthy (e.g., injured, diseased, disrupted) patient, according to an embodiment.

FIG. 8 illustrates a flowchart of another method for diagnosing the physiological state of the patient, according to an embodiment.

FIG. 9 illustrates a visual representation of the ultrasound data, according to an embodiment.

FIG. 10A illustrates a graph showing frequency and/or velocity versus time using spectral Doppler audio data captured from a healthy vessel, and FIG. 10B illustrates a graph showing frequency and/or velocity versus time using spectral Doppler audio data captured from a non-healthy vessel, according to an embodiment.

FIG. 11 illustrates an output (e.g., graph) showing acoustic biomarkers for the healthy vessel and non-healthy vessel, according to an embodiment.

FIG. 12 illustrates a flowchart of another method for diagnosing the physiological state of the patient, according to an embodiment.

FIG. 13 illustrates a graph showing portions that have been extracted from the spectral Doppler audio data, according to an embodiment.

FIG. 14 illustrates a schematic view of the extracted portions shifting due to changes in the blood flow, according to an embodiment.

FIG. 15 illustrates a schematic view of an output showing a probability that the vessel is not healthy, according to an embodiment.

### DETAILED DESCRIPTION

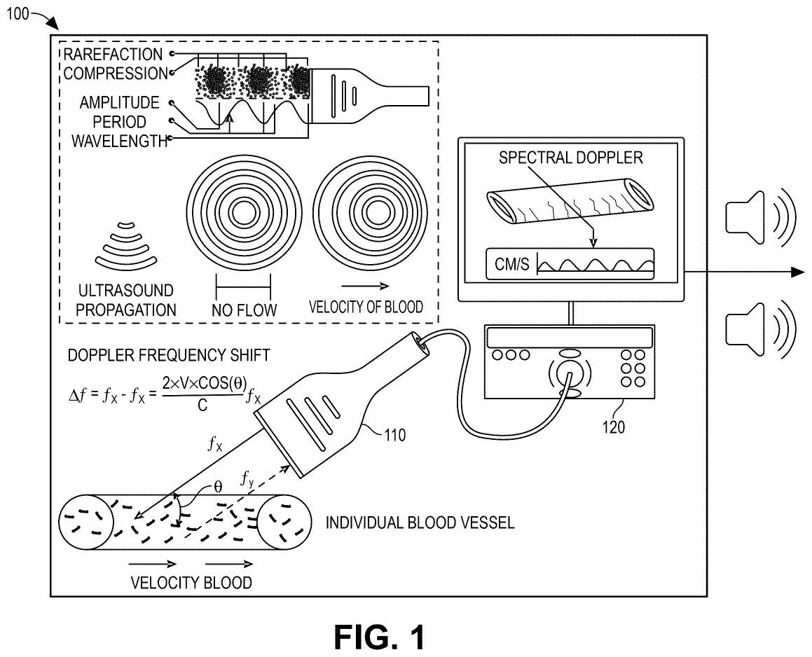

FIG. 1 illustrates a schematic view of a system 100 for processing ultrasound data, according to an embodiment. The system 100 may include a sensor 110 that is configured to capture ultrasound data from the patient. The sensor 110 may be or include (or be part of) a handheld ultrasound transducer, a continuously wearable device (e.g., an armband, bracelet, adhesive patch, etc.), an implantable device, an ingestible pill, or a combination thereof. The system 100 may also include a computing system 120. The computing system 120 may be configured to receive the ultrasound data, process the ultrasound data, and generate an output based upon the processed ultrasound data. The output may be used to diagnose a physiological state of the patient.

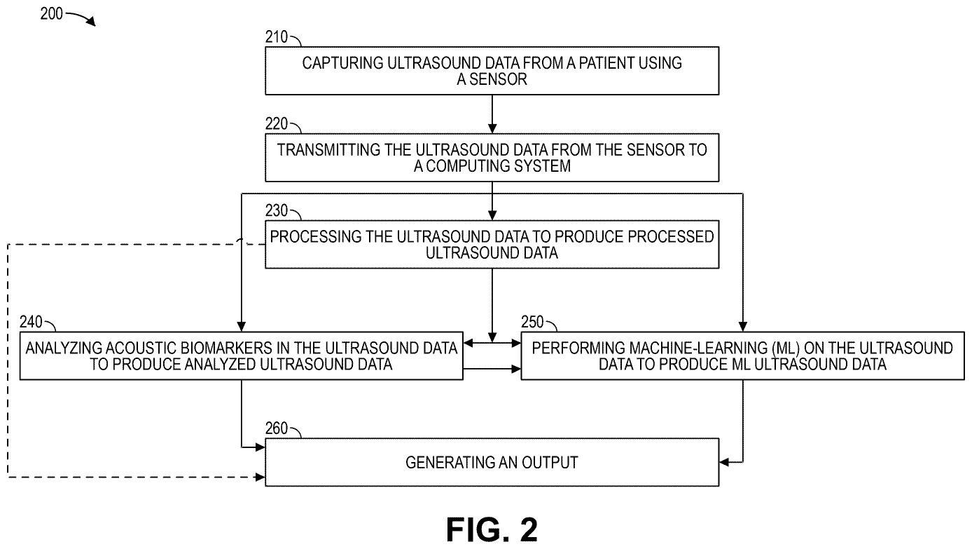

FIG. 2 illustrates a flowchart of a method 200 for diagnosing a physiological state of the patient, according to an embodiment. An illustrative order of the method 200 is provided below; however, one or more steps of the method 200 may be performed in a different order, simultaneously, repeated, or omitted. One or more steps of the method 200 may be performed by the system 100.

The method 200 may include capturing ultrasound data from a patient using the sensor 110, as at 210. The ultrasound data may be or include raw (e.g., unfiltered) ultrasound audio data. The ultrasound data may also or instead be or include element or channel data that is transformed and still includes all of the data/information. The ultrasound data also or instead may be or include ultrasound audio data such as spectral Doppler data. The ultrasound data may be captured from a single patient or a plurality of different patients. The ultrasound data may also or instead be captured at a single time or at a plurality of different times. The ultrasound data may also or instead be captured for a single organ or a plurality of different organs. The ultrasound data may also or instead be captured at a single location in the organ or at a plurality of different locations in/along the organ. The organ may be or include a brain, a spinal cord, a heart, a liver, a kidney, a bladder, an artery, a vein, or a combination thereof.

The method 200 may also include transmitting the ultrasound data from the sensor 110 to the computing system 120, as at 220. The transmission may be through a wire or wirelessly.

The method 200 may also include processing the ultrasound data to produce processed ultrasound data, as at 230. The ultrasound data may be processed using the computing system 120. In one embodiment, the ultrasound data may be processed by performing a spatial and/or temporal transformation on the ultrasound data. In another embodiment, the ultrasound data may be processed by combining (e.g., fusing) ultrasound modalities. As used herein, an ultrasound modality may include A-mode, M-mode, B-mode, spectral Doppler ultrasound, and the like. For example, ultrasound audio data and ultrasound image data may be combined (e.g., fused). In another embodiment, the ultrasound data may be processed by combining (e.g., fusing) acoustic features in the ultrasound data. As used herein, an acoustic feature refers to direct or indirect feature engineering. The acoustic features may be or include any biomarkers. The acoustic features may include at least part of the data that helps to determine a clinical/physiological state (e.g., injured, turbulent flow, etc.). The processed ultrasound data may be in a readable and/or compatible file type.

The method 200 may also include analyzing acoustic biomarkers in the ultrasound data to produce analyzed ultrasound data, as at 240. As used herein, an acoustic biomarker refers to an acoustic feature or any transformation from audio data that indicates/yields biological and/or clinical information. The acoustic biomarkers may be analyzed in the raw ultrasound data (e.g., from step 210 or 220) and/or in the processed ultrasound data (e.g., from step 230). In one embodiment, the acoustic biomarkers may be analyzed by comparing the ultrasound data to previously-captured ultrasound data in a database (e.g., of the computing system 120). More particularly, this may include comparing the acoustic biomarkers in the ultrasound data to corresponding acoustic biomarkers in the previously-captured ultrasound data in the database. The previously-captured ultrasound data in the database (and/or the acoustic biomarkers therein) may be previously-determined to be from a healthy patient and/or organ (e.g., vessel) in the patient, an injured patient and/or organ in the patient, a diseased patient and/or organ in the patient, a disrupted patient and/or organ in the patient, or a combination thereof.

In another embodiment, the acoustic biomarkers may be analyzed by combining the processed audio data. For example, the processed audio data at the different times, different organs, and/or different locations (e.g., in the same organ) may be combined.

The method 200 may also include performing machine-learning (ML) on the ultrasound data to produce ML ultrasound data, as at 250. The ML may be performed on the raw ultrasound data (e.g., from step 210 or 220), the processed ultrasound data (e.g., from step 230), the analyzed ultrasound data (e.g., from step 240), or a combination thereof. The ML may include classification, grading, and/or ranking the ultrasound data with existing and/or adapted pipelines. In one example, the ML may include uniform manifold approximation and projection (UMAP). In another example, the ML may include t-distributed stochastic neighbor embedding (t-SNE).

The method 200 may also include generating an output, as at 260. The output may be based at least partially upon the raw ultrasound data (e.g., from step 210 or 220), the processed ultrasound data (e.g., from step 230), the analyzed ultrasound data (e.g., from step 240), the ML ultrasound data (e.g., from step 250), or a combination thereof. The output may provide a clinically relevant insight about the patient. In one embodiment, the output may provide a visualization (e.g., graph) of the health of the patient. In another embodiment, the output may provide audio playback and/or feedback about the patient. In another embodiment, the output may provide tactile signal generation for the patient. In another embodiment, the output may provide binary classification of a healthy versus perturbed state of the patient. As used herein, a perturbed state refers to any state that is not baseline or changes from the baseline state. Examples may include injuries, blood pressure changes, turbulent flow, blood clot formations, or a combination thereof.

In another embodiment, the output may provide predictive and/or probabilistic evaluation (e.g., diagnostics) for the patient. For example, the output may diagnose a physiological state of the patient. The physiological state may be or include healthy, injured, diseased, disrupted, or a combination thereof. The physiological state may also or instead include the location of the injury, disease, disruption, or a combination thereof. The physiological state may also or instead include the degree of the injury, disease, disruption, or a combination thereof. As used herein, “disruption” refers to features found in the audio data as a result of some biological/clinical change (e.g., blood pressure, bleeding, viscosity).

In another embodiment, the output may provide mesh signals that may be used to reconstruct a predicted occlusion or state. In another embodiment, the output may alert a medical professional of a particular biomarker or state of the patient. In another embodiment, the output may provide vibrational feedback to notify a medical professional that intervention is recommended. In another embodiment, the output may indicate the presence of a disease or state in the patient. In another embodiment, the output may identify trends and/or progressions that may indicate a possible disruption in the patient.

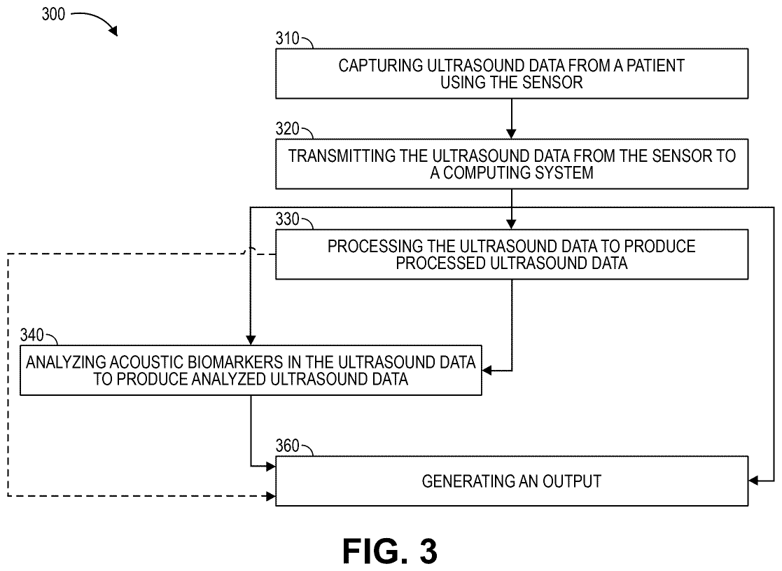

FIG. 3 illustrates a flowchart of another method 300 for diagnosing the physiological state of the patient, according to an embodiment. An illustrative order of the method 300 is provided below; however, one or more steps of the method 300 may be performed in a different order, simultaneously, repeated, or omitted. One or more steps of the method 300 may be performed by the system 100. Some portions of the method 300 may be similar to the method 200 and, for brevity, may not be described again in detail below.

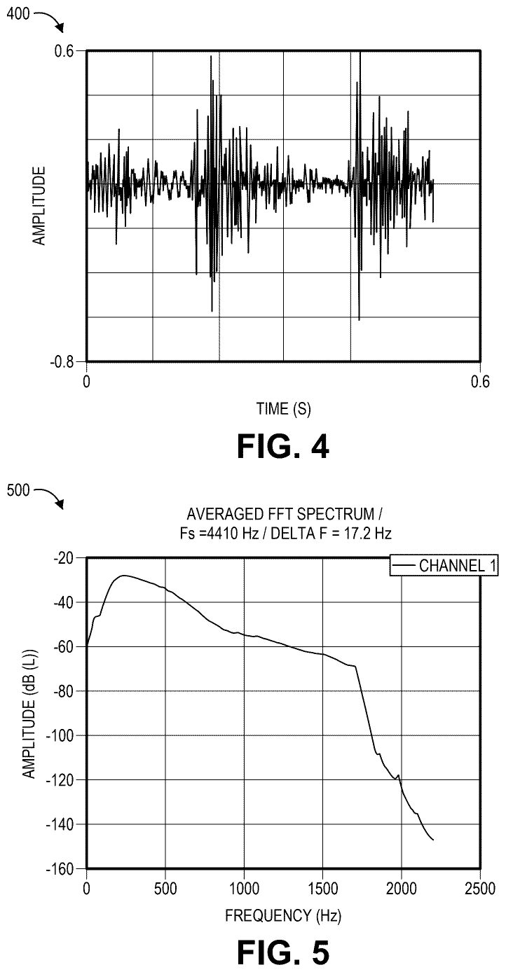

The method 300 may include capturing ultrasound data from a patient using the sensor 110, as at 310. The ultrasound data may be or include raw (e.g., unfiltered) ultrasound audio data. The ultrasound data may also or instead be or include ultrasound audio data such as spectral Doppler data. FIG. 4 illustrates a graph 400 showing spectral Doppler audio data, according to an embodiment. The X axis represents time, and the Y axis represents amplitude.

The method 300 may also include transmitting the ultrasound data from the sensor 110 to the computing system 120, as at 320.

The method 300 may also include processing the ultrasound data to produce processed ultrasound data, as at 330. The ultrasound data may be processed using the computing system 120. The ultrasound data may be processed by performing a spatial and/or temporal transformation on the ultrasound data. FIG. 5 illustrates a graph (e.g., periodogram) 500 showing the spectral Doppler audio data after a spatial and/or temporal frequency transformation, according to an embodiment. The X axis represents time, and the Y axis represents amplitude.

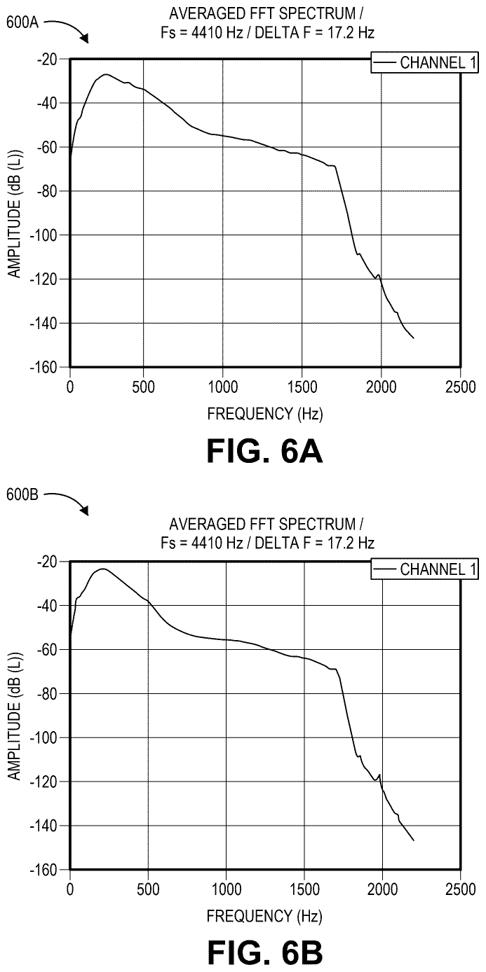

The method 300 may also include analyzing acoustic biomarkers in the ultrasound data to produce analyzed ultrasound data, as at 340. The acoustic biomarkers may be analyzed in the processed ultrasound data (e.g., from step 330). The acoustic biomarkers may be analyzed by combining the processed audio data. FIGS. 6A and 6B illustrate a plurality of graphs (e.g., periodograms) 600A, 600B that may be combined, according to an embodiment. In the example shown, the graphs 600A, 600B represent different parts of the same organ at the same time. For example, the graphs 600A, 600B may represent two different axial locations along the spinal cord at the same time. In another example, the graphs 600A, 600B may represent the same organ and/or the same part of the organ at two different times.

The method 300 may also include generating an output, as at 360. The output may be based at least partially upon the raw ultrasound data (e.g., from step 310 or 320), the processed ultrasound data (e.g., from step 330), the analyzed ultrasound data (e.g., from step 340), or a combination thereof. The output may provide a visualization (e.g., graph) of the health of the patient. The output may also or instead provide mesh signals that may be used to reconstruct a predicted occlusion or state. The output may also or instead alert a medical professional of a particular biomarker or state of the patient.

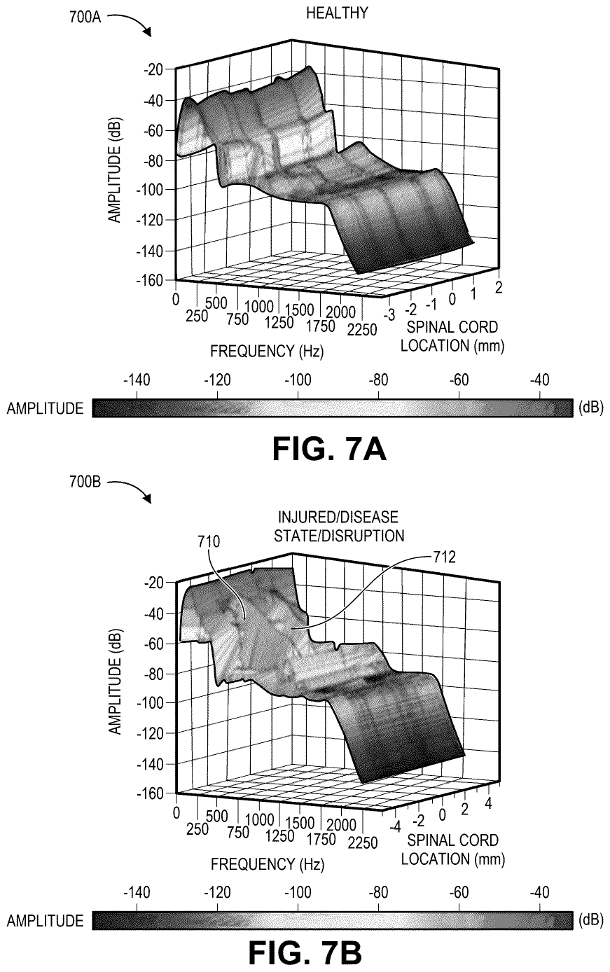

FIG. 7A illustrates an output (e.g., a 3D audio-frequency graph) 700A for a healthy patient, according to an embodiment. The graph 700A represents a plurality of graphs (e.g., including the graphs 600A, 600B) shown side-by-side. FIG. 7B illustrates an output (e.g., a 3D audio-frequency graph) 700B for a non-healthy (e.g., injured, diseased, disrupted) patient, according to an embodiment. The two raised areas 710, 712 in the graph 700B represent the injured, diseased, and/or disrupted locations on the organ (e.g., spinal cord). In the graphs 700A, 700B, one axis represents the location (e.g., along the spinal cord), one axis represents frequency, and one axis represents amplitude. Thus, the location, frequency, and amplitude of the non-healthy locations 710, 712 can be identified.

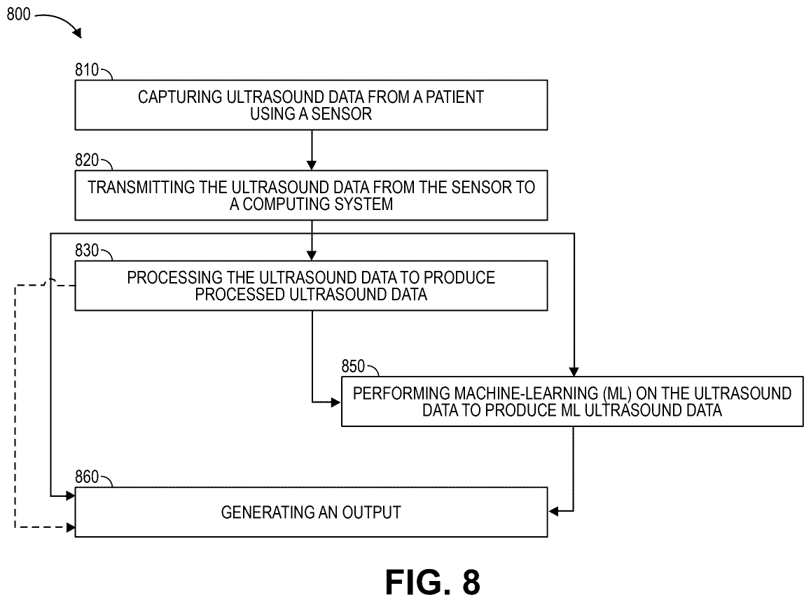

FIG. 8 illustrates a flowchart of another method 800 for diagnosing the physiological state of the patient, according to an embodiment. An illustrative order of the method 800 is provided below; however, one or more steps of the method 800 may be performed in a different order, simultaneously, repeated, or omitted. One or more steps of the method 800 may be performed by the system 100. Some portions of the method 800 may be similar to the method(s) 200, 300 and, for brevity, may not be described again in detail below.

The method 800 may include capturing ultrasound data from a patient using the sensor 110, as at 810. The ultrasound data may be or include raw (e.g., unfiltered) ultrasound audio data. The ultrasound data may also or instead be or include element or channel data that is transformed while still maintaining all of the data. The ultrasound data also or instead may be or include ultrasound audio data such as spectral Doppler data.

The method 800 may also include transmitting the ultrasound data from the sensor 110 to the computing system 120, as at 820.

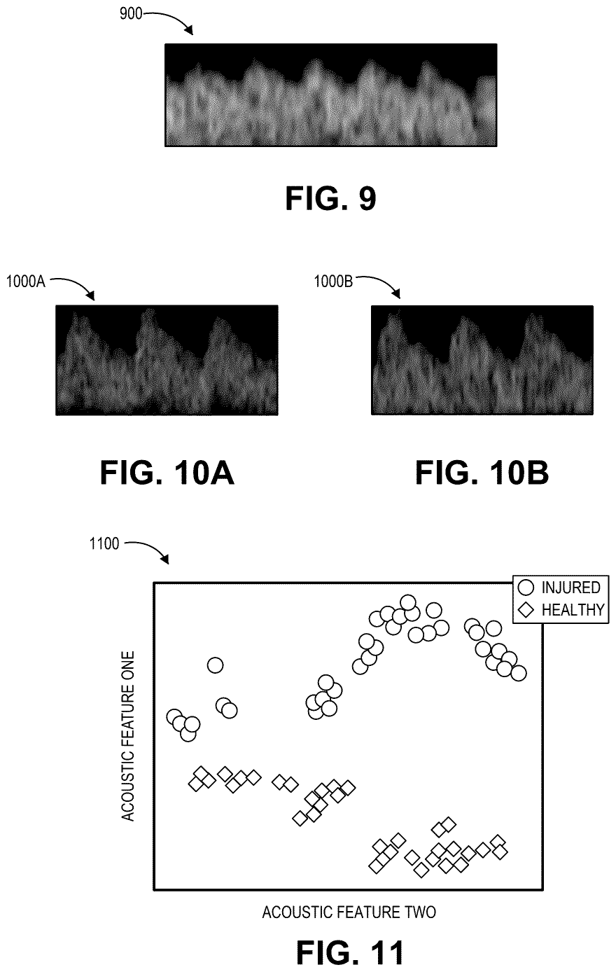

The method 800 may also include processing the ultrasound data to produce processed ultrasound data, as at 830. FIG. 9 illustrates a visual representation 900 of the processed ultrasound data, according to an embodiment.

The method 800 may also include performing machine-learning (ML) on the ultrasound data to produce ML ultrasound data, as at 850. The ML may be performed on the raw ultrasound data (e.g., from step 810 or 820) and/or the processed ultrasound data (e.g., from step 830). The ML may include classification, grading, and/or ranking the ultrasound data with existing and/or adapted pipelines. In one example, the ML may include uniform manifold approximation and projection (UMAP). In another example, the ML may include t-distributed stochastic neighbor embedding (t-SNE).

FIGS. 10A and 10B illustrate ML ultrasound data. More particularly, FIG. 10A illustrates a graph 1000A showing frequency and/or velocity versus time using spectral Doppler audio data captured from a healthy organ (e.g., vessel), and FIG. 10B illustrates a graph 1000B showing frequency and/or velocity versus time using spectral Doppler audio data captured from a non-healthy (e.g., injured, diseased, disrupted) organ, according to an embodiment. As will be appreciated, it may be difficult to visually identify any differences between the graphs 1000A, 1000B.

The method 800 may also include generating an output, as at 860. The output may be based at least partially upon the raw ultrasound data (e.g., from step 810 or 820), the processed ultrasound data (e.g., from step 830), the ML ultrasound data (e.g., from step 850), or a combination thereof.

FIG. 11 illustrates an output (e.g., graph) 1100 showing acoustic biomarkers (e.g., the dots on the graph 1100) for the healthy organ and non-healthy organ, according to an embodiment. The raw audio signals may be feed into ML pipelines such as UMAP and t-SNE to generate the output. Acoustic features may (e.g., directly) enable the clustering to one or more biomarkers. The output (e.g., graph) 1100 may provide binary classification of a healthy versus non-healthy (e.g., diseased) state of the organ and/or patient. In the graph 1100, the X axis represents a first acoustic feature, and the Y axis represents a second acoustic feature. The audio data is compressed into two acoustic features that best represent and/or differentiate subgroups. This may be referred to as dimensionality reduction.

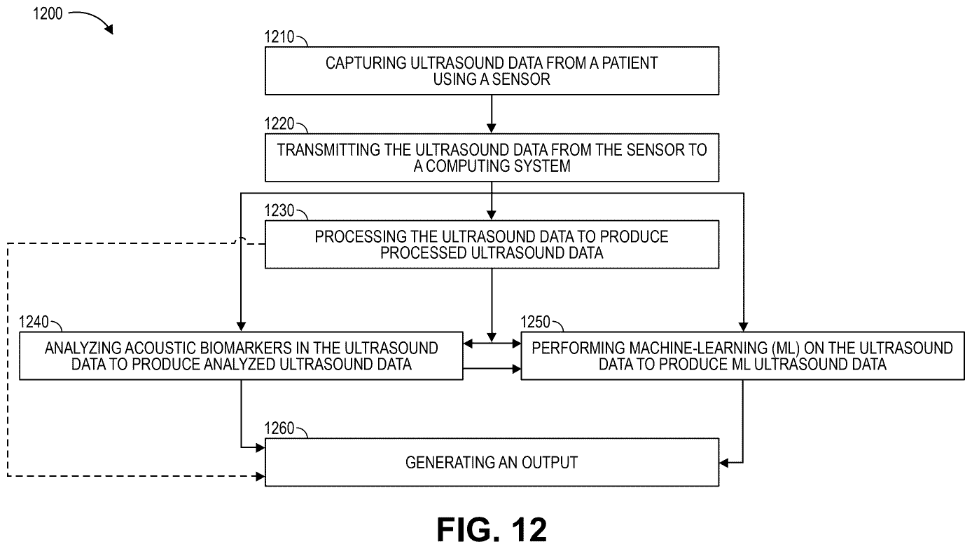

FIG. 12 illustrates a flowchart of another method 1200 for diagnosing the physiological state of the patient, according to an embodiment. An illustrative order of the method 1200 is provided below; however, one or more steps of the method 1200 may be performed in a different order, simultaneously, repeated, or omitted. One or more steps of the method 1200 may be performed by the system 100. Some portions of the method 1200 may be similar to the method(s) 200, 300, 800 and, for brevity, may not be described again in detail below.

The method 1200 may include capturing ultrasound data from a patient using the sensor 110, as at 1210. The ultrasound data may be or include raw (e.g., unfiltered) ultrasound audio data. The ultrasound data also or instead may be or include ultrasound audio data such as spectral Doppler data.

The method 1200 may also include transmitting the ultrasound data from the sensor 110 to the computing system 120, as at 1220.

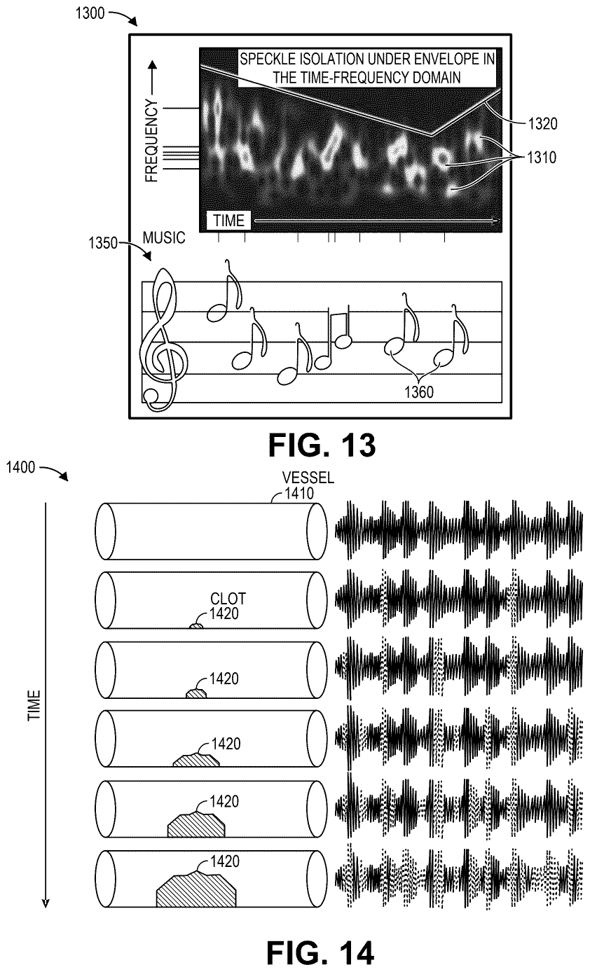

The method 1200 may also include processing the ultrasound data to produce processed ultrasound data, as at 1230. The ultrasound data may be processed using the computing system 120. In one embodiment, the ultrasound data may be processed by performing a spatial and/or temporal transformation on the ultrasound data. FIG. 13 illustrates a graph 1300 showing the processed ultrasound data, according to an embodiment. More particularly, the graph 1300 shows portions that have been extracted from the spectral Doppler audio data. In the graph 1300, the speckles 1310 represent regions of high-density, signal origin isolation via a wavelet and/or superlet processing. In the graph 1300, the line 1320 above the speckles represents the envelope of the signal. The method analyzes the envelope and the signal underneath. The X axis represents time, and the Y axis represents frequency.

FIG. 13 also illustrates music 1350 that has been generated to correspond to the graph 1300. More particularly, the music includes musical notes 1360 that correspond to the speckles 1310 in the graph 1300. The regions of high density and/or signal origin can be represented or played as musical notes at the corresponding frequency, amplitude, time points, or a combination thereof. This may be done for real-time audio feedback or for a continuation of audio processing. The real-time audio feedback may be a pronounced, loud, and/or distinguishable musical note which alerts of a physiological state. A harmony, or spectrum of notes, may also indicate a physiological state. Moreover, a perturbation or lack thereof of an expected note (i.e., syncopation) may indicate a physiological change.

The method 1200 may also include analyzing acoustic biomarkers in the ultrasound data to produce analyzed ultrasound data, as at 1240. The acoustic biomarkers may be analyzed in the raw ultrasound data (e.g., from step 1210 or 1220) and/or in the processed ultrasound data (e.g., from step 1230). The acoustic biomarkers may be analyzed by combining the processed audio data (e.g., in the graph 1300 and/or music 1350). For example, the processed audio data from different times, different organs, and/or different locations (e.g., in the same organ) may be combined.

FIG. 14 illustrates a schematic view of the analyzed (e.g., combined) ultrasound data, according to an embodiment. More particularly, FIG. 14 shows the extracted portions (e.g., speckles and/or line) 1310, 1320 shifting due to changes in the blood flow, according to an embodiment. More particularly, the left side of FIG. 14 shows an organ (e.g., a vessel) 1410 with a blockage (e.g., clot) 1420 that increases in size over time. The right side of FIG. 14 shows the audio signal continues to have disruptions detected in the audio as the blood clot 1420 grows. The turbulent or disrupted flow is captured by the audio. The profile of Doppler shifts from red blood cells alter the acoustic profile.

The method 1200 may also include performing machine-learning (ML) on the ultrasound data to produce ML ultrasound data, as at 1250. The ML may be performed on the raw ultrasound data (e.g., from step 1210 or 1220), the processed ultrasound data (e.g., from step 1230), the analyzed ultrasound data (e.g., from step 1240), or a combination thereof. The ML data may include classification, grading, and/or ranking the ultrasound data with existing and/or adapted pipelines. In one example, the ML may include uniform manifold approximation and projection (UMAP). In another example, the ML may include t-distributed stochastic neighbor embedding (t-SNE).

The method 1200 may also include generating an output, as at 1260. The output may be based at least partially upon the raw ultrasound data (e.g., from step 1210 or 1220), the processed ultrasound data (e.g., from step 1230), the analyzed ultrasound data (e.g., from step 1240), the ML ultrasound data (e.g., from step 1250), or a combination thereof. The output may provide predictive and/or probabilistic evaluation (e.g., diagnostics) for the patient. For example, the output may diagnose a physiological state of the patient. In another embodiment, the output may identify trends and/or progressions that may indicate a possible disruption in the patient. For example, the output may identify the blood clot 1420 with 98% certainty, identify the size of the blood clot 1420, identify whether the blood clot 1420 is increasing or decreasing in size over time, and provide a recommendation such as “drink more water.”

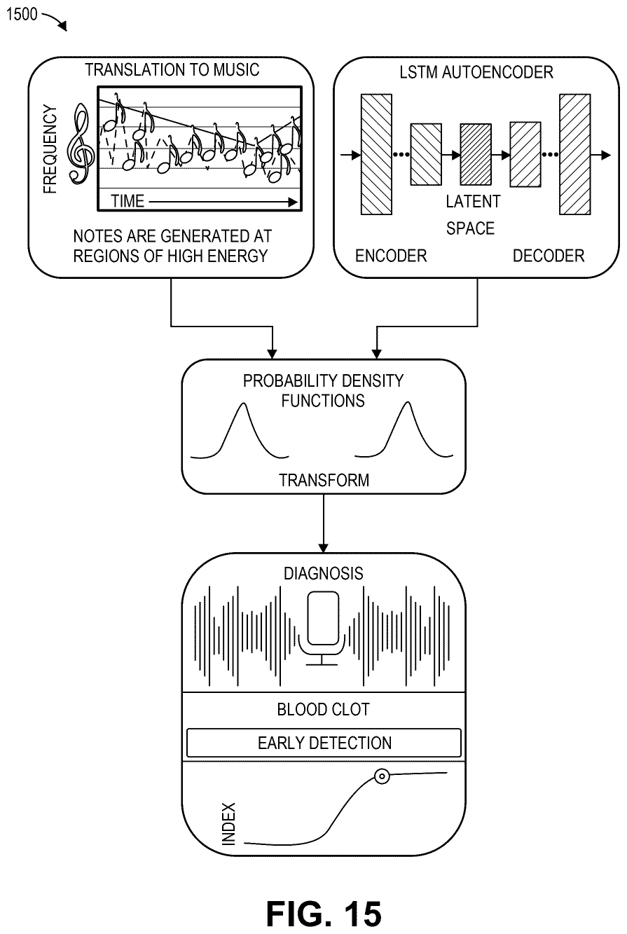

FIG. 15 illustrates a schematic view of an output 1500 showing a probability that the vessel 1410 is not healthy, according to an embodiment. FIG. 15 shows possible pipelines for an automated diagnosis. The autoencoder and/or probability density functions can be utilized to inform about the physiological state. The neural networks processing and/or statistical analysis and interpretations may be dependent on the physiological state in focus. The raw or processed data may be transformed through this pipeline to predict or diagnose a physiological state (e.g., with an index of certainty).

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. A method for diagnosing a physiological state of a patient, the method comprising:
capturing ultrasound audio data in the patient using a sensor;
processing the ultrasound audio data to produce processed ultrasound audio data; and
generating an output based at least partially upon the processed ultrasound audio data, wherein the output provides the physiological state of the patient, wherein the physiological state of the patient comprises a certainty of an existence of a non-healthy region in the patient at a plurality of different times and a location of the non-healthy region in the patient at the different times.

2. The method of claim 1, wherein processing the ultrasound audio data comprises:
performing a spatial transformation and/or a temporal transformation on the ultrasound audio data; and
generating a periodogram based upon the ultrasound audio data after the spatial transformation and/or the temporal transformation is performed.

3. The method of claim 1, wherein processing the ultrasound audio data comprises combining different ultrasound modalities of the ultrasound audio data, and wherein the ultrasound modalities comprise A-mode, M-mode, B-mode, spectral Doppler ultrasound, or a combination thereof.

4. The method of claim 1, wherein processing the ultrasound audio data comprises combining different acoustic features in the ultrasound audio data.

5. The method of claim 1, further comprising analyzing acoustic biomarkers in the processed ultrasound audio data to produce analyzed ultrasound audio data, wherein analyzing the acoustic biomarkers in the processed ultrasound audio data comprises combining two or more graphs containing the processed ultrasound audio data, wherein each of the two or more graphs corresponds to the same part of an organ the at the different times or different parts of the organ at a same time, and wherein the output is based at least partially upon the analyzed ultrasound audio data.

6. The method of claim 5, wherein the output comprises a 3D audio-frequency graph comprising a combination of the two or more graphs, and wherein axes of the 3D audio-frequency graph comprise the location, a frequency, and an amplitude.

7. The method of claim 1, further comprising performing machine learning (ML) on the processed ultrasound audio data to produce ML ultrasound audio data, wherein the ML comprises classification, grading, ranking, uniform manifold approximation and projection (UMAP), t-distributed stochastic neighbor embedding (t-SNE), or a combination thereof.

8. The method of claim 1, wherein the output comprises a graph showing acoustic biomarkers in the processed ultrasound audio data.

9. The method of claim 8, wherein the graph comprises binary classification, which identifies each of the acoustic biomarkers as either healthy or non-healthy.

10. The method of claim 9, wherein the ultrasound audio data comprises more than two acoustic features, wherein processing the ultrasound audio data comprises performing dimensionality reduction to reduce a number of the acoustic features down to two acoustic features that best represent the ultrasound audio data, wherein a first axis of the graph represents a first of the two acoustic features, and wherein a second axis of the graph represents a second of the two acoustic features.

11. A method for diagnosing a physiological state of a patient, the method comprising:
capturing ultrasound audio data in the patient using a sensor, wherein the ultrasound audio data comprises spectral Doppler audio data of an organ in the patient;
processing the ultrasound audio data to produce processed ultrasound audio data, wherein processing the ultrasound audio data comprises performing a spatial transformation and/or a temporal transformation on the ultrasound audio data to produce a graph;
analyzing acoustic biomarkers in the processed ultrasound audio data to produce analyzed ultrasound audio data, wherein the acoustic biomarkers are analyzed by combining the processed ultrasound audio data from different times;
generating images of the organ the different times;
performing machine learning (ML) on the analyzed ultrasound audio data to produce ML ultrasound audio data, wherein the ML comprises classification, grading, ranking, uniform manifold approximation and projection (UMAP), t-distributed stochastic neighbor embedding (t-SNE), or a combination thereof; and
generating an output based at least partially upon the ML ultrasound audio data, wherein the output provides the physiological state of the patient, wherein the physiological state of the patient comprises a certainty of an existence of a non-healthy region at the different times and a location of the non-healthy region at the different times.

12. The method of claim 11, wherein the graph is in a time-frequency domain.

13. The method of claim 12, wherein the graph comprises an envelope of the ultrasound audio data and speckles below the envelope.

14. The method of claim 13, wherein the speckles represent regions of high-density, signal origin isolation in the ultrasound audio data.

15. The method of claim 11, wherein the physiological state of the patient also comprises a trend or a progression of the non-healthy region the different times and at future times.

16. A method for diagnosing a physiological state of a patient, the method comprising:
capturing ultrasound audio data from the patient using a sensor, wherein the ultrasound audio data comprises spectral Doppler audio data;
processing the ultrasound audio data to produce processed ultrasound audio data, wherein processing the ultrasound audio data comprises performing a spatial transformation and a temporal transformation on the ultrasound audio data to produce a graph in a time-frequency domain, wherein the graph comprises an envelope of the ultrasound audio data and speckles below the envelope, wherein the speckles represent regions of high-density, signal origin isolation in the ultrasound audio data;
analyzing acoustic biomarkers in the graph to produce analyzed ultrasound audio data, wherein the acoustic biomarkers are analyzed in the envelope and in the speckles below the envelope, wherein the acoustic biomarkers are analyzed by combining the processed ultrasound audio data from different times, different locations in a same organ, different organs, or a combination thereof;
generating images of the same organ and/or the different organs at the different times to show a disruption;
performing machine learning (ML) on the images to produce ML ultrasound audio data, wherein the ML comprises classification, grading, ranking, uniform manifold approximation and projection (UMAP), t-distributed stochastic neighbor embedding (t-SNE), or a combination thereof; and
generating an output based at least partially upon the ML ultrasound audio data, wherein the output provides the physiological state of the patient, wherein the physiological state of the patient comprises a certainty of an existence of the disruption at the different times, a size of the disruption at the different times, the location of the disruption at the different times, and a trend or a progression of the disruption at the different times and at future times.

17. The method of claim 16, further comprising generating musical notes that correspond the speckles.

18. The method of claim 17, wherein the musical notes correspond to a time, a frequency, and an amplitude of the speckles.

19. The method of claim 16, wherein the disruption comprises a blood clot.

20. The method of claim 16, wherein the output provides predictive and/or probabilistic evaluation of the physiological state of the patient.
